(19) United States
(12) Patent Application Publication
(10) Pub. No.: US 2022/0031605 A1
(43) Pub. Date: Feb. 3, 2022

(54) TARGETED DRUG DELIVERY SYSTEM TO BE USED IN TREATING OSTEOMYELITIS

(71) Applicant: EGE UNIVERSITESI, Izmir (TR)

(72) Inventors: Senay SANLIER, Izmir (TR); Guliz AK, Izmir (TR); Habibe YILMAZ, Izmir (TR); Ummuhan Fulden BOZKAYA, Izmir (TR); Ozge SARI, Izmir (TR)

(73) Assignee: EGE UNIVERSITESI, Izmir (TR)

(21) Appl. No.: 17/278,467

(22) PCT Filed: Sep. 23, 2019
(86) PCT No.: PCT/TR2019/050786

§ 371 Date: Mar. 22, 2021

(30) Foreign Application Priority Data
Sep. 26, 2018 (TR) 2018/13983

(51) Int. Cl. A61K 9/00; A61K 31/7036; A61K 9/51

(52) U.S. Cl. CPC A61K 9/0009; A61K 9/5192; A61K 9/5169; A61K 31/7036

(57) ABSTRACT

A targeted drug delivery system to be used in treating osteomyelitis is provided. Gelatin nanoparticles suitable for a drug release in treating the osteomyelitis includes magnetite and antibiotics, wherein the antibiotics are gentamicin. A production method of the gelatin nanoparticles includes the steps of: dissolving collagen, enabling a precipitation of gelatin having a large mass of molecule, removing the gelatin having a small sized molecule mass, re-dissolving the gelatin with the large mass of the molecule in water and adjusting a pH of the gelatin to 10-13 with a NaOH solution after the dissolving is completed, adding 2-6 mg/ml magnetite dispersion, adding acetone dropwise, adding 0.02-0.2 ml 5-9 mg/ml genipin as a cross linker, and adding 3.5-7 mg/ml 0.1-1 ml antibiotics on magnetic gelatin nanoparticles.

### CROSS REFERENCE TO THE RELATED APPLICATIONS

This application is the national stage entry of International Application No. PCT/TR2019/050786, filed on Sep. 23, 2019, which is based upon and claims priority to Turkish Patent Application No. 2018/13983, filed on Sep. 26, 2018, the entire contents of which are incorporated herein by reference.

### TECHNICAL FIELD

The invention is related to a targeted drug delivery system to be used in treating osteomyelitis.

### BACKGROUND

Osteomyelitis is bone inflammation due to an organism that causes infection. Although under normal conditions the bone tissue is resistant against bacterial colonization, traumas or surgical operations, presence of foreign matter or prosthesis etc., may cause bone inflammation to be induced. Moreover osteomyelitis may lead to hematogenous propagation. The incidence of spinal osteomyelitis has been determined to be 1 in 450000 in the year 2001. However it is believed that this rate will increase due to reasons such as, population age increase, and intravenous drug usage in the future years. The total osteomyelitis incidence in developed countries is much higher.

Early and specific antibiotic treatment is quite important in osteomyelitis.

Many patients receive at least 4 to 6 weeks of antibiotic treatment following the removal of dead bone tissue with a surgical operation. This process leads to drug toxicity in the patient and may cause resistance to antibiotics and repetition of the infection.

By developing biodegradable and magnetic targeted antibiotics delivery system for treating osteomyelitis, drug resistance and toxicity is reduced and besides this, therapeutic efficiency is increased.

### SUMMARY

The system subject to the invention provides targeted delivery of antibiotics by means of the magnetic nanoparticles it contains. By this means drug toxicity and drug resistance arising from high dose is prevented.

Gelatin which is a natural polymer, was converted into form of nanoparticles and was encapsulated therein with magnetic nanoparticles for magnetic targeting and the drug (antibiotic) was absorbed into the gelatin nanoparticles. The nanocarrier system is biodegradable because of its gelatin structure, and is also directed to the infected area by means of the magnetic field applied externally with the help of the magnetic properties it has gained due to its magnetite content. The drug that is adsorbed due to both the magnetic targeting the gelatin nanoparticle and due to the physical nature of the drug, does not remain in circulation for a long period of time and is not released immediately into healthy tissues. However, due to the decrease in the pH of the infected tissue, the gelatin nanoparticle-drug interaction is interrupted; therefore the drug which is drawn to the infected area is released from the drug delivery system. The controlled release of this drug is provided. It is believed that by this means the drug amount injected and injection time can be reduced, which is also very important for the well being of the patient.

### BRIEF DESCRIPTION OF THE DRAWINGS

FIG. 1: Transmission Electron Microscopy (TEM) imaging belonging to Magnetic Gelatin Nanoparticles comprising Gentamicin

FIG. 2: Zeta Size Analysis Result belonging to Magnetic Gelatin Nanoparticles comprising Gentamicin

FIG. 3A: Drug release graphic belonging to free form (commercial) gentamicin

FIG. 3B: Drug release graphic belonging to the Magnetic Gelatin Nanoparticles comprising Gentamicin

FIG. 4A: Group I: Radiograph image taken 14 days after the osteomyelitis model is established regarding the magnetic gelatin nanoparticle group comprising gentamicin

FIG. 4B: Group I: Radiograph image taken after 6 doses of therapy regarding the magnetic gelatin nanoparticle group comprising gentamicin

FIG. 4C: Group II: Radiograph image taken 14 days after the osteomyelitis model is established for the free gentamicin group.

FIG. 4D: Group II: Radiograph image taken 6 days after therapy for the free gentamicin group

FIG. 4E: Group III: Radiograph image taken 14 days after the osteomyelitis model is established for the control group.

FIG. 4F: Group III: Radiograph image taken after 6 doses of therapy for the control group

### DETAILED DESCRIPTION OF THE EMBODIMENTS

By means of the invention, antibiotics are loaded to a biodegradable and biocompatible delivery system and are carried; and time controlled release from this system is provided, additionally it is aimed to reduce drug toxicity and prevent resistance to antibiotics by physically targeting the drug encapsulated in the system by means of magnetite, to the infected tissue.

### Synthesis of Magnetic Gelatin Nanoparticles Comprising Gentamicin

Collagen is formed from type B gelatin produced with alkali hydrolysis and is dissolved in 35-65 mg water. Following this 0.5-2 ml acetone is rapidly added and the precipitation of gelatin having a large mass of molecule is enabled. The gelatin having small sized molecule mass that has not precipitated and remains as supernatant is removed from the medium. The gelatin with large molecule mass is re-dissolved in water. After dissolving is completed pH is adjusted to 10-13 with NaOH solution and 2-6 mg/ml magnetite dispersion is added.

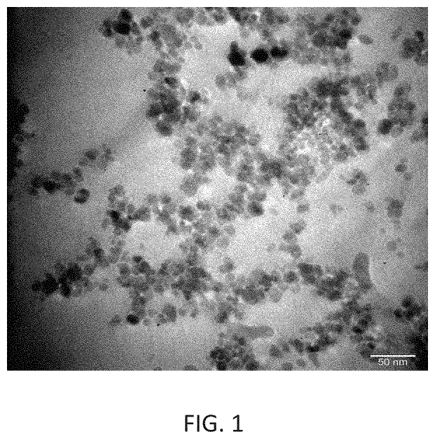

In order to form gelatin nanoparticles containing magnetite, acetone was added dropwise onto the mixture at 0.5-2 ml/min. In order to establish nanoparticle stabilization of the formed gelatin, 0.02-0.2 ml 5-9 mg/ml genipin was added as cross linker and incubation was carried out for 5-7 hours at room temperature. After stable magnetic nanoparticles were obtained, centrifuge and washing is carried out in order to remove acetone from the medium. On top of the magnetic gelatin nanoparticles that have been dispersed in water 3.5-7 mg/ml 0.1-1 ml gentamicin is added and incubation is carried out at 30-40° C. for 12-20 hours in order to perform drug adsorption. The non adsorbed drug, is separated from the drug loaded nanoparticles by centrifuge. The amount of the loaded drug, is calculated by means of UV-spectroscopy over the drug amount that has been separated by centrifuge, that has remained as supernatant and has not been loaded. The characterization of the drug loaded magnetic gelatin nanoparticles has been carried out with Zeta size analysis and transmission electron microscopy (TEM) (FIG. 1).

It has been determined that the nanoparticles were almost spherical and that their dry form was approximately 20-30 nm.

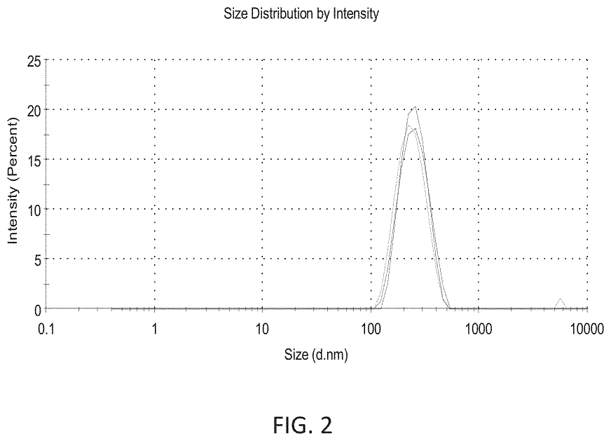

The sizes of the nanoparticles in aqueous medium was calculated as 253.7 nm by means of zeta size analysis (FIG. 2).

### In Vitro Drug Release

The release profile of the drug adsorbed to magnetic gelatin nanoparticles and 0.1-1 mg free drug was examined. In order to carry this out nanoparticles comprising free drugs and gentamicin have been subjected to dialysis at 30-40° C. against a 5-20 ml pH 6-8 phosphate buffer. The buffer was changed at certain times and the amount of drug released was determined by means of UV spectroscopy.

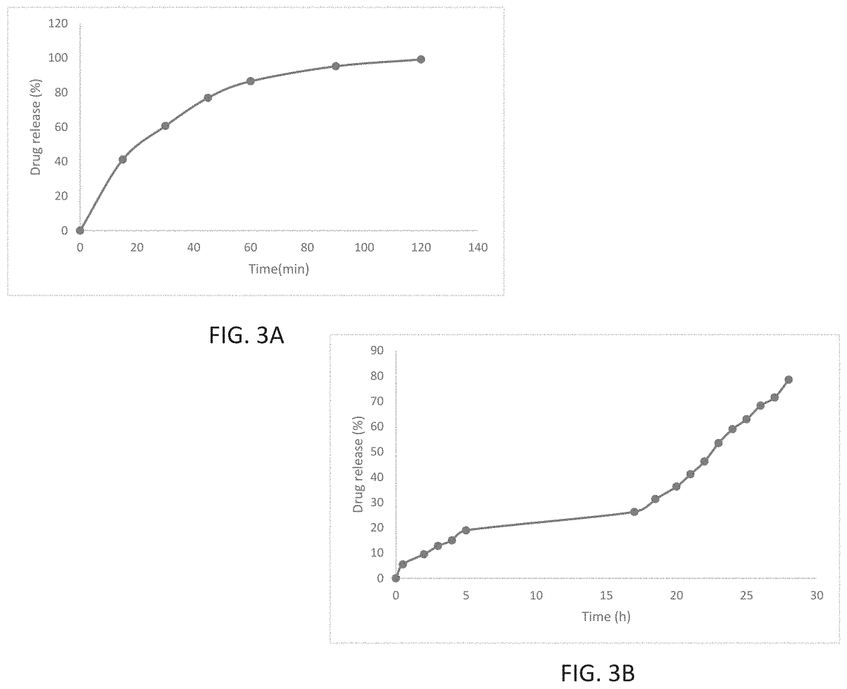

It has been observed that the drug release of the nanoparticles was controlled in comparison to free form drugs (FIGS. 3A-3B).

### In Vivo Trials

Wistar albino rats were operated on, and osteomyelitis model was established by injecting Staphylococcus Aureus pathogen to the proximal tibia. In order to determined that the osteomyelitis model was formed, Xray (Radiograph) image was taken. The animals that were determined to have osteomyelitis, were separated into 3 groups, as the gelatin nanoparticle comprising gentamicin (I), free drug (II) and control (III) groups, and they were included in the treatment with 2-4 injections a weak. 100-300 μl, gelatin nanoparticle comprising gentamicin, free form (commercial gentamicin and physiological saline solution was injected intravenously into the tail vein of the groups I, II, and III respectively. On the 14th day following the commencement of treatment, a radiograph was taken in order to track the treatment process. Hematological analysis was carried out during the treatment and after the treatment.

The lymphocyte reference range for rats was 1.4-9.4×10³/μL.

As a result of the haemogram carried out on healthy rats, the lymphocyte value was 7.4×10³/μL.

| | Lymphocyte | Lymphocyte |
|---|---|---|
| | number | number |
| | following 6 | of following |
| | dose stherapy | 11 doses of |
| Groups | (10³/μL) | therapy (10³/μL) |
| A Magnetic Gelatin Nanoparticle | 10.6 | 9.2 |
| Group comprising Gentamicin | | |
| (group I) | | |
| Free gentamicin group (group II) | 12.6 | Could not be |
| | | determined. |
| Control Group (group III) | 11.2 | Could not be |
| | | determined. |

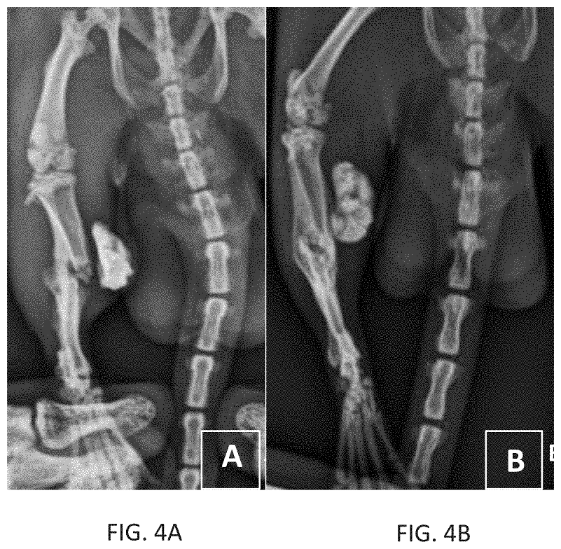

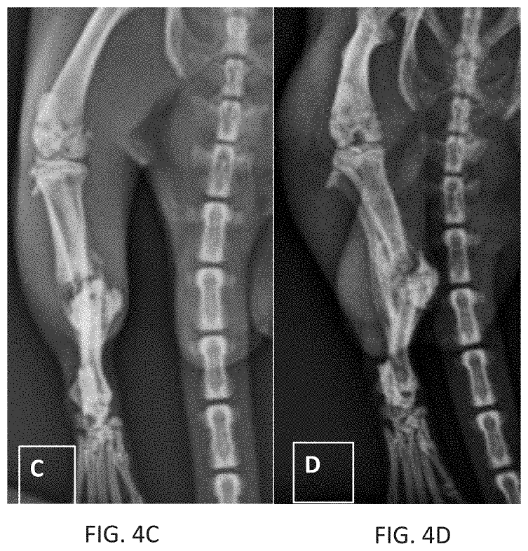

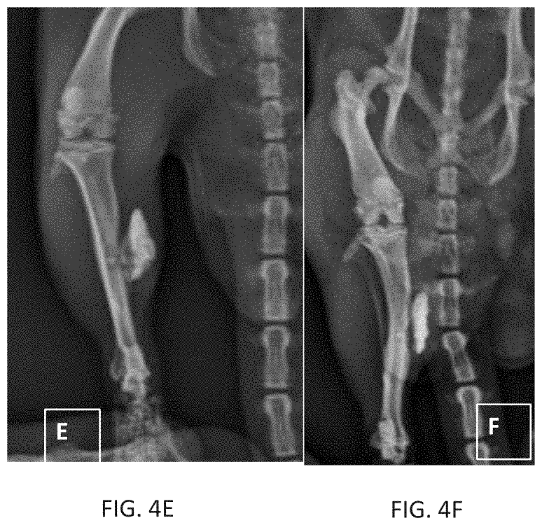

As it can be seen in FIG. 4A, while abscess formation and bone integrity degradation is present after 14 days of establishing an osteomyelitis model, it can be seen in FIG. 4B that the abscess had improved and the periost and bone integrity had started to be reformed following 6 doses of therapy with the magnetic gelatin nanoparticle comprising gentamicin of the invention.

In FIG. 4C, according to the radiograph image taken after 14 days of the establishment of the osteomyelitis model, abscess formation was present and the bone integrity was degraded, however in FIG. 4D, it can be observed that following the application of free gentamicin, abscess was present, bone integrity was not yet formed and healing was only present on the periost.

In FIG. 4E, according to the radiograph image taken after 14 days of the establishment of the osteomyelitis model, abscess formation was present and the bone integrity was degraded, however in FIG. 4F, any kind of healing or improvement was not observed and abscess was also present in the control group following 6 doses of therapy.

What is claimed is:

1. Gelatin nanoparticles suitable for a drug release in treating osteomyelitis, comprising magnetite and antibiotics.

2. The gelatin nanoparticles according to claim 1, wherein the antibiotics are gentamicin.

3. A production method of the gelatin nanoparticles according to claim 1, comprising the steps of:
i. dissolving collagen formed from a type B gelatin produced with an alkali hydrolysis in 35-65 mg of water;
ii. enabling a precipitation of the type B gelatin having a large mass of molecule with an addition of 0.5-2 ml acetone;
iii. removing the type B gelatin having a small sized molecule mass, wherein the type B gelatin has not precipitated and remains as a supernatant from a medium;
iv. re-dissolving the type B gelatin with the large mass of the molecule in water and adjusting a pH of the type B gelatin to 10-13 with a NaOH solution after the dissolving is completed;
v. adding 2-6 mg/ml magnetite dispersion;
vi. adding acetone dropwise to form the gelatin nanoparticles containing the magnetite;
vii. adding 0.02-0.2 ml 5-9 mg/ml genipin as a cross linker to establish a gelatin nanoparticle stabilization and carrying out a first incubation for 5-7 hours at room temperature; and
viii. adding 3.5-7 mg/ml 0.1-1 ml antibiotics on magnetic gelatin nanoparticles, wherein the magnetic gelation nanoparticles have been dispersed in water and carrying out a second incubation at 30-40° C. for 12-20 hours to perform a drug adsorption.

4. The production method according to claim 3, wherein the antibiotics are gentamicin.
